(19) United States
(12) Patent Application Publication
(10) Pub. No.: US 2026/0306572 A1
(43) Pub. Date: Oct. 8, 2026

(54) Aqueous Dispersions of Hydrophobic Dietary Ingredients and Methods of Preparation

(71) Applicants: Anneliese Hodgson, Fullerton, CA (US); Donald Hodgson, Fullerton, CA (US)

(72) Inventors: Anneliese Hodgson, Fullerton, CA (US); Donald Hodgson, Fullerton, CA (US)

(21) Appl. No.: 19/636,913

(22) Filed: Apr. 2, 2026

Related U.S. Application Data
(60) Provisional application No. 63/783,173, filed on Apr. 3, 2025.

(51) Int. Cl. A61K 36/54; A23L 2/38; A23L 2/56; A23L 2/58; A23L 27/10; A23L 33/21; A61K 9/10; A61K 47/14; A61K 47/24; A61K 47/26; A61K 47/36; A61K 47/42

(52) U.S. Cl. CPC A61K 36/54; A23L 2/38; A23L 2/56; A23L 2/58; A23L 27/10; A23L 33/21; A61K 9/10; A61K 47/14; A61K 47/24; A61K 47/26; A61K 47/36; A61K 47/42

(57) ABSTRACT

The present invention relates to compositions and methods for producing a drinkable aqueous dispersion of hydrophobic dietary ingredients, particularly whole-milled cinnamon, to enhance its dispersibility, bioavailability, and palatability. The invention overcomes the limitations of conventional cinnamon formulations by incorporating a triglyceride oil as a milling aid and dispersibility enhancer, in conjunction with a surfactant system, to facilitate wet bead milling and achieve submicron or nanoscale particle sizes. The resulting dispersion retains the complete bioactive profile of whole cinnamon, including fiber, essential oils, and polyphenols, while significantly improving aqueous compatibility and sensory characteristics. The formulation ensures long-term suspension stability, prevents sedimentation, and enables efficient absorption of bioactive compounds, making it suitable for incorporation into functional foods, nutraceuticals, and pharmaceutical applications.

### BACKGROUND OF THE INVENTION

### Field of Invention

The present invention relates to compositions and methods for enhancing the aqueous dispersibility of hydrophobic dietary ingredients. More particularly, the invention pertains to the formulation of stable aqueous dispersions of such ingredients through the incorporation of oil and surfactant systems, followed by particle size reduction via wet bead milling. The invention further relates to applications in the food, nutraceutical, and pharmaceutical industries, wherein improved solubility, bioavailability, and organoleptic properties of bioactive compounds are critical to efficacy and consumer acceptance.

### Brief Summary of the Invention

The present invention provides compositions and methods for producing aqueous dispersions of hydrophobic dietary ingredients, specifically cinnamon, to enhance their dispersibility, bioavailability, and palatability. The formulation comprises a hydrophobic dietary ingredient combined with a triglyceride oil and optionally a surfactant, followed by particle size reduction via wet bead milling. This process yields fine dispersions with improved wettability, facilitating incorporation into aqueous consumables while preserving the biological activity of the dietary ingredient.

The invention addresses key challenges associated with hydrophobic dietary ingredients, including poor water solubility, undesirable mouthfeel, and limited bioavailability. By reducing particle size and leveraging oil-surfactant interactions, the invention enables effective delivery of bioactive compounds, potentially improving metabolic health outcomes such as blood glucose regulation and insulin sensitivity. The disclosed methods and compositions are applicable across various industries, including functional foods, nutraceuticals, and pharmaceuticals.

### BRIEF DESCRIPTION OF THE FIGURES

FIG. 1: The process of producing a stable aqueous cinnamon dispersion begins with ingredient selection and pre-mixing (101), followed by preliminary high-shear mixing (103) to ensure uniform distribution, after which wet bead milling (105) reduces particle size to a submicron or nanoscale range, with subsequent stabilization and homogenization (107) to prevent re-agglomeration, quality control and analytical testing (109) to verify composition and stability, final packaging and storage (111) to maintain product integrity, and integration into consumer products (113) for use in functional foods, nutraceuticals, and pharmaceutical formulations.

### DETAILED DESCRIPTION

The present invention relates to a novel composition and method for producing a drinkable aqueous dispersion of hydrophobic dietary ingredients, specifically whole-milled cinnamon, to enhance its dispersibility, bioavailability, and palatability. Cinnamon is widely recognized for its potential health benefits, including its role in glucose metabolism, insulin sensitivity, cardiovascular health, and inflammation modulation. However, its hydrophobic nature, poor wettability, and fibrous structure present significant challenges in formulating an easily consumable liquid product that delivers its full biological activity in a palatable form. This invention overcomes these limitations by employing a proprietary wet milling process, in combination with triglyceride oils and optionally surfactants, to create a smooth, stable, and bioavailable aqueous cinnamon dispersion.

Traditional cinnamon formulations, such as dry powders, capsules, and extracts, fail to adequately address issues related to water dispersibility, texture, and bioavailability. Ground cinnamon is difficult to consume in clinically relevant doses due to its poor solubility and its tendency to cause an unpleasant drying effect in the mouth. The challenges of ingesting dry cinnamon were demonstrated in viral “Cinnamon Challenge” videos, where participants attempted to swallow a spoonful of cinnamon powder and experienced extreme difficulty due to its hydrophobic nature and fine particulate structure. Additionally, cinnamon extracts may provide a concentrated form of certain bioactive compounds, but they do not retain the full spectrum of beneficial components found in whole cinnamon, such as fiber and essential oils. The present invention preserves the complete nutritional and bioactive profile of whole cinnamon while transforming it into a readily dispersible and palatable liquid form.

The aqueous dispersion formulation consists of several essential components that work synergistically to improve stability, texture, and bioavailability. The primary ingredient is whole-milled cinnamon, which provides a full spectrum of bioactive compounds, including cinnamaldehyde, fiber, essential oils, tannins, and polyphenols. Unlike cinnamon extracts that isolate individual compounds, this formulation retains both the soluble and insoluble fractions of cinnamon, maximizing prebiotic, metabolic, and anti-inflammatory benefits. The cinnamon particles are reduced to a submicron or nanoscale size, with a mean particle diameter of less than 20 microns (D50), significantly improving their dispersibility in aqueous solutions and enhancing absorption in the gastrointestinal tract.

A key aspect of the invention is the incorporation of triglyceride oils as a milling aid and dispersibility enhancer. Unlike conventional wet milling, which typically relies solely on surfactants to stabilize aqueous dispersions, this invention uniquely incorporates triglyceride oils to facilitate the size reduction process while also enhancing the stability of the final dispersion. Suitable triglyceride oils include medium-chain triglycerides (MCTs), long-chain triglycerides (LCTs), and various plant-derived oils. The use of triglyceride oils in wet milling is a novel and unexpected approach, as oils are inherently hydrophobic and do not function as traditional surfactants. However, it has been discovered that the presence of triglyceride oils in the milling process unexpectedly enhances particle size reduction, prevents excessive heat generation, and preserves the integrity of bioactive compounds. This contributes to the overall fluidity of the dispersion and ensures a smooth, non-gritty texture upon consumption.

The formulation also includes a surfactant system designed to reduce interfacial tension between the hydrophobic cinnamon particles and the aqueous phase, thereby maintaining long-term suspension stability. Suitable surfactants include lecithins, phospholipids, polysorbates, and protein-based emulsifiers. The selection of an appropriate surfactant or surfactant blend is based on its hydrophilic-lipophilic balance (HLB) value, which must be carefully optimized to achieve the desired dispersion characteristics. The surfactant system prevents particle aggregation and sedimentation, ensuring that the cinnamon remains evenly dispersed throughout the aqueous medium without requiring excessive agitation or reconstitution before consumption.

The preparation of the aqueous dispersion follows a controlled multi-step process to achieve optimal particle size, bioavailability, and long-term stability. First, the organic whole-milled cinnamon, triglyceride oil, and surfactant are combined in specific ratios to form a coarse pre-mix. This pre-mixing step ensures that the hydrophobic cinnamon particles are pre-coated with dispersion aids before undergoing mechanical size reduction. Next, the formulation is subjected to high-energy wet bead milling, a process in which ceramic, zirconia, or polymer beads exert shear forces on the particles, breaking them down to a submicron or nanoscale range. The milling parameters, including bead size, milling duration, and temperature, are carefully controlled to optimize particle size uniformity while preventing the degradation of volatile essential oils and other bioactive compounds. The use of triglyceride oils as a fluidizing agent during the milling process provides a unique advantage by reducing friction, minimizing heat generation, and maintaining the structural integrity of key bioactive compounds within the cinnamon.

Following the milling process, the dispersion undergoes a stabilization phase to prevent re-agglomeration of the particles and ensure long-term homogeneity. This step may involve additional emulsification techniques or the incorporation of co-stabilizers such as natural gums, proteins, or viscosity modifiers that enhance the stability of the dispersion over extended storage periods. The final product is a pourable, stable aqueous dispersion that can be easily incorporated into beverages, liquid supplements, pharmaceutical suspensions, and other water-based consumer products without requiring additional mixing or emulsification steps.

The present invention offers numerous advantages over conventional cinnamon formulations and other delivery methods for hydrophobic dietary ingredients. Unlike dry cinnamon powders, which exhibit poor water dispersibility and an undesirable mouthfeel, the disclosed formulation provides a smooth, easily consumable liquid dispersion that retains the complete nutritional and functional properties of whole cinnamon. The wet milling process ensures that the cinnamon particles are sufficiently small to eliminate grittiness, while the incorporation of triglyceride oils and surfactants results in a stable suspension that remains homogenous without phase separation.

One of the most significant benefits of this invention is the enhancement of bioavailability. The reduction of particle size increases the overall surface area of the cinnamon particles, leading to improved dissolution rates and more efficient absorption in the gastrointestinal tract. This results in higher bioavailability of key bioactive compounds, such as cinnamaldehyde, polyphenols, and fiber-based prebiotics, which contribute to improved metabolic health outcomes. Preliminary studies suggest that twice-daily consumption of this aqueous dispersion may lead to improved insulin sensitivity and better blood glucose control in individuals with type 2 diabetes and prediabetic conditions. Additionally, the formulation has potential applications in cardiovascular health by promoting cholesterol reduction and supporting vascular function.

Beyond cinnamon, the disclosed formulation and processing method can be adapted to a wide range of other hydrophobic dietary ingredients, including botanical extracts, lipid-soluble vitamins, polyphenolic antioxidants, and other bioactive compounds with poor aqueous solubility. The versatility of this invention extends its applicability to functional foods, nutraceuticals, and pharmaceuticals, where aqueous dispersibility and enhanced bioavailability are critical to product efficacy. The disclosed methods can also be further optimized by adjusting milling parameters, surfactant compositions, and oil selection based on the specific characteristics of the dietary ingredient being processed.

While wet bead milling is the preferred technique for particle size reduction, alternative mechanical processes, such as high-pressure homogenization, microfluidization, or ultrasonic processing, may be employed under specific conditions to achieve similar results. Furthermore, the formulation may be modified to include additional stabilizing agents or functional excipients, depending on the intended application of the dispersion. These modifications provide flexibility in adapting the invention to different dietary and pharmaceutical formulations, ensuring that the disclosed methods can be broadly applied across various product categories.

The present invention represents a significant advancement in the formulation and delivery of hydrophobic dietary ingredients, offering a novel approach to improving aqueous dispersibility, bioavailability, and sensory characteristics. By addressing the challenges associated with poor solubility, difficult mouthfeel, and limited absorption, this invention provides a practical and effective solution for incorporating whole-milled cinnamon and other hydrophobic bioactive compounds into liquid-based consumer products. The disclosed methods and compositions have broad applicability in the fields of nutrition, health, and medicine, with the potential to enhance the functional benefits of dietary ingredients and improve consumer compliance.

### DETAILED DESCRIPTION OF FIGURES

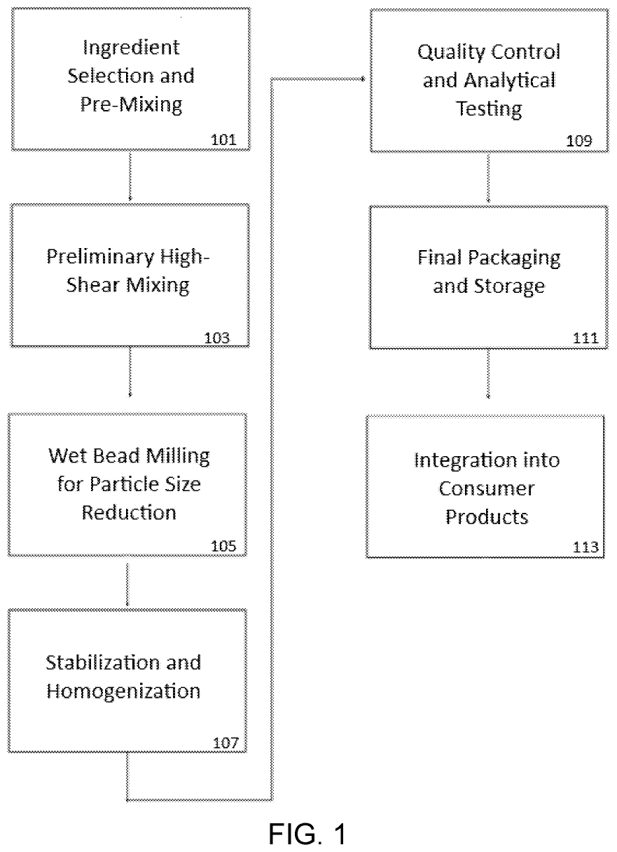

### FIG. 1.101—Ingredient Selection and Pre-Mixing

The process begins with the selection of high-quality organic whole-milled cinnamon, a triglyceride oil, and a surfactant system. The cinnamon is screened for purity, moisture content, and particle size consistency. The selected triglyceride oil, which serves as a milling aid and dispersion stabilizer, is measured precisely based on the target viscosity and processing parameters. The surfactant system, chosen based on its hydrophilic-lipophilic balance (HLB), is incorporated to facilitate dispersion. These components are combined in predetermined ratios to create a coarse pre-mix, ensuring uniform distribution of the oil and surfactant over the cinnamon particles before mechanical size reduction.

### FIG. 1.103—Preliminary High-Shear Mixing

The pre-mix is subjected to mixing and optionally in some cases high-shear mixing using a rotor-stator system to achieve preliminary homogenization. This step enhances the distribution of the triglyceride oil and surfactant around the cinnamon particles, reducing clumping and preparing the mixture for efficient milling. High-shear mixing generates strong mechanical forces that break apart initial aggregates, ensuring that all particles are evenly coated with the dispersion aids. The process is carefully controlled to prevent excessive heat buildup, which could degrade the volatile essential oils within the cinnamon.

### FIG. 1.105—Wet Bead Milling for Particle Size Reduction

The homogenized mixture is fed into a wet bead milling system, where it undergoes high-energy mechanical size reduction. The milling chamber contains ceramic, zirconia, or polymer beads that exert shear and impact forces on the cinnamon particles, breaking them down to a submicron or nanoscale range. The milling conditions, including bead size, agitation speed, and processing duration, are optimized to achieve a particle size distribution with a mean diameter (D50) of less than 20 microns. Temperature control is maintained to prevent oxidation and preserve the integrity of bioactive compounds such as cinnamaldehyde and polyphenols.

### FIG. 1.107—Stabilization and Homogenization

After wet milling, the dispersion is subjected to a stabilization process to prevent particle re-agglomeration and ensure long-term suspension stability. Additional emulsifiers or viscosity modifiers may be introduced to enhance the uniformity of the dispersion. High-pressure homogenization is optionally employed to refine the texture further, eliminating any residual coarse particles and improving mouthfeel. The stabilized dispersion is evaluated for viscosity, flow characteristics, and sensory properties to confirm that it meets formulation specifications.

### FIG. 1.109—Quality Control and Analytical Testing

The aqueous dispersion undergoes rigorous quality control testing to ensure compliance with safety, stability, and regulatory standards. Particle size distribution is analyzed using laser diffraction, while zeta potential measurements confirm dispersion stability. Microbiological assays are performed to detect contaminants, ensuring that the product meets USP <51> antimicrobial effectiveness standards. Chemical analysis verifies the concentration of key bioactive compounds, including cinnamaldehyde, polyphenols, and essential oils. Accelerated stability testing is conducted to assess the long-term shelf life of the formulation under various environmental conditions.

### FIG. 1.111—Final Packaging and Storage

The finished dispersion is transferred into pre-sterilized, food-grade containers designed for long-term stability. The packaging format is selected based on the intended application, with options including multi-dose bottles, single-serving sachets, or bulk storage containers for industrial use. If required, the product undergoes aseptic filling to maintain sterility. Labels are applied with nutritional information, storage instructions, and usage guidelines. The final packaged product is stored under controlled temperature and humidity conditions to preserve its stability and efficacy.

### FIG. 1.113—Integration into Consumer Products

The aqueous cinnamon dispersion is incorporated into functional beverages, dietary supplements, and pharmaceutical formulations for targeted health applications. It can be used as a base ingredient for ready-to-drink products, meal replacements, or metabolic health formulations. Additional flavoring agents, sweeteners, or complementary nutraceuticals such as phytosterols or curcumin may be blended to enhance consumer appeal. The product is distributed through retail, e-commerce, and healthcare channels, ensuring accessibility for consumers seeking metabolic and cardiovascular health benefits.

### Example Use Case

A male subject with type 2 diabetes for 9 years (diagnosed in 2017 at age 61) was administered milled cinnamon 15 mL twice daily equivalent to 3 g Ceylon cinnamon.

Prior to consuming the encapsulated, surface modified, milled cinnamon the subject routinely consumed 1 tablespoon of ground Ceylon cinnamon once daily before bed and administered 60 u of insulin aspart (15 u with breakfast, 17 u with lunch and 26-28 u with dinner) daily, together with 500 mg metformin twice daily, and a GLP1 agonist once weekly for a period of more than one year.

After 3 months of consuming encapsulated milled cinnamon the subject noted more than a dozen incidents of low blood sugar that required immediate action, this, in-turn, required the subject to reduce administered insulin aspart required to maintain normal blood glucose to 15 u daily (5 u with breakfast, 5 u with lunch and 5 u with dinner) while maintaining other medication doses during the treatment period. The average daily blood glucose prior to consuming the milled cinnamon product was 150 mg/dL, which decreased to 125 mg/dL after 6 months use, corresponding HbA1c went from 6.9 at the beginning of the treatment period to 6.0 after 6 months.

These data indicate that the milled cinnamon product provided a significant improvement in insulin sensitivity as demonstrated by the reduced daily insulin aspart dose required to maintain a healthy blood glucose level.

What is claimed is:

1. A composition for forming an aqueous dispersion of a hydrophobic dietary ingredient, comprising:
a) whole-milled cinnamon as the hydrophobic dietary ingredient;
b) a triglyceride oil as a milling aid and dispersion stabilizer;
c) a surfactant system for emulsification; and
d) an aqueous medium,
wherein the composition is processed via wet bead milling to reduce the particle size to a submicron or nanoscale range, thereby enhancing dispersibility, bioavailability, and palatability of the hydrophobic dietary ingredient.

2. A method for preparing a stable aqueous dispersion of a hydrophobic dietary ingredient, comprising:
a) providing whole-milled cinnamon;
b) combining the cinnamon with a triglyceride oil and a surfactant system to form a pre-mix;
c) subjecting the pre-mix to wet bead milling under controlled temperature conditions to reduce the particle size to a submicron or nanoscale range; and
d) dispersing the milled composition in an aqueous medium, wherein the resulting dispersion exhibits improved aqueous compatibility, long-term stability, and enhanced bioavailability of the hydrophobic dietary ingredient.

3. The aqueous dispersion prepared according to the method of claim 2, wherein the final dispersion maintains homogeneity and remains free from phase separation or sedimentation for a period of at least six months under ambient storage conditions.

4. The composition of claim 1, wherein the triglyceride oil is selected from the group consisting of medium-chain triglycerides (MCTs), long-chain triglycerides (LCTs), or plant-derived triglyceride oils.

5. The composition of claim 1, wherein the surfactant system is selected from the group consisting of lecithins, phospholipids, polysorbates, protein-based emulsifiers, or combinations thereof.

6. The method of claim 2, wherein the wet bead milling is performed using ceramic, zirconia, or polymer bead media to achieve a particle size distribution with a D50 of less than 20 microns.

7. The method of claim 2, wherein the milling process is conducted under low-temperature conditions to prevent oxidation and preserve volatile essential oils.

8. The aqueous dispersion of claim 3, wherein the composition maintains enhanced bioavailability by increasing the intestinal absorption rate of cinnamaldehyde and polyphenols.

9. The method of claim 2, further comprising the addition of stabilizing agents selected from the group consisting of natural gums, proteins, or viscosity modifiers to further enhance dispersion stability.

10. The method of claim 2, wherein the aqueous dispersion is formulated for incorporation into a functional beverage, dietary supplement, or pharmaceutical suspension.

11. The composition of claim 1, wherein the aqueous dispersion is free from synthetic preservatives or organic solvents.

12. The method of claim 2, wherein the dispersion is optimized for prebiotic effects, promoting gut microbiome health by providing insoluble and soluble dietary fiber.

13. The method of claim 2, wherein the dispersion is designed for metabolic health applications, including blood glucose regulation, insulin sensitivity improvement, and cholesterol management.

14. The composition of claim 1, wherein the resulting aqueous dispersion has a smooth, non-gritty texture, improving consumer acceptability and compliance.

15. The method of claim 2, wherein alternative mechanical size reduction techniques selected from the group consisting of high-pressure homogenization, microfluidization, or ultrasonic processing may be used in place of wet bead milling.

16. The method of claim 2, wherein the formulation is adaptable for additional flavoring or functional ingredient inclusion, such as phytosterols, chromium picolinate, curcumin, or non-nutritive sweeteners.
